(19) United States
(12) Patent Application Publication
(10) Pub. No.: US 2022/0306598 A1
(43) Pub. Date: Sep. 29, 2022

(54) METHOD FOR HIGHLY SELECTIVE CONVERSION OF CBD TO DELTA-8 THC

(71) Applicant: PRECISION EXTRACTION CORPORATION, Troy, MI (US)

(72) Inventor: Dmitry Kuklev, Ann Arbor, MI (US)

(21) Appl. No.: 17/212,173

(22) Filed: Mar. 25, 2021

(51) Int. Cl. C07D 311/78

(52) U.S. Cl. CPC C07D 311/78

(57) ABSTRACT

A cannabis conversion method includes providing a starting material that includes cannabidiol (CBD), combining the starting material with a solvent and a Lewis acid to form a mixture, the solvent and the Lewis acid reacting with the CBD to form a product Delta-8-tetrahydrocannabinol in the mixture, and removing the solvent and the Lewis acid from the mixture, the removing leaving the product Delta-8-tetrahydrocannabinol.

### BACKGROUND

Cannabinoids derived from cannabis plants are desired for a variety of therapeutic uses. The cannabinoids are present as cannabinolic acids in cannabis plant material, such as tetrahydrocannabinolic acid and cannabidiolic acid. The cannabinolic acids and other substances are extracted from the plant material using known extraction processes. Subsequently, the cannabinolic acids are subjected to a decarboxylation process to form their conjugate cannabinoids, such as cannabidiol (CBD) and tetrahydrocannabinol (THC).

CBD can also be converted to various forms of THC, such as Delta-9-THC, Delta-8-THC, and iso-THC. CBD has the chemical formula 1,3-Benzenediol, 2-[R1R,6R)-3-methyl-6-(1-methylethenyl)-2-cyclohexen-1-yl]-5-pentyl and Delta-9-THC has the chemical formula 6aR,7,10,10aR-tetrahydro-6,6,9-trimethyl-3-pentyl-6H-dibenzo[b,d]pyran-1-ol, the molecules of which are shown below for reference. Structurally, the CBD molecule differs from the Delta-9-THC by an open ring in the CBD molecule that is a closed ring in the Delta-9-THC (i.e., CBD is bicyclic and THC is tricyclic). The CBD can be converted to Delta-9-THC in a cyclization reaction to close the ring. Such processes, however, often produce low yields of conversion and low purity product that has byproduct isomer molecules, such as Delta-8-THC and iso-THC. In turn, the byproducts necessitate that the product be further processed by high performance liquid chromatography (HPLC) to isolate the desired Delta-8-THC from its isomers.

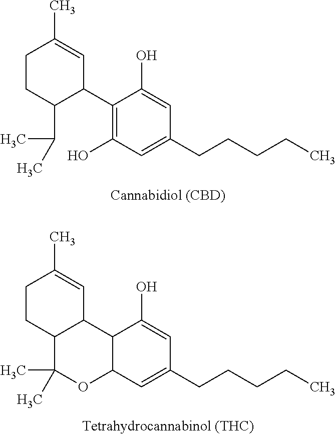

### SUMMARY

A cannabis conversion method according to an example of the present disclosure includes providing a starting material that includes cannabidiol (CBD), and combining the starting material with a solvent and a Lewis acid to form a mixture. The solvent and the Lewis acid react with the CBD to form a product Delta-8-tetrahydrocannabinol in the mixture. The solvent and the Lewis acid are then removed from the mixture, leaving the product Delta-8-tetrahydrocannabinol.

In a further embodiment of any of the foregoing embodiments, the solvent is selected from the group consisting of hexane, heptane, cyclohexane, isooctane, tolulene, benzene, and combinations thereof.

In a further embodiment of any of the foregoing embodiments, the Lewis acid is selected from the group consisting of perchloric acid, sulfuric acid, alkylsulfonic acid, methane sulfonic acid, arylsulfonic acid, p-toluene sulfonic acid, and combinations thereof.

In a further embodiment of any of the foregoing embodiments, the solvent is a halogenated hydrocarbon selected from the group consisting of dichloromethane, chloroform, carbon tetrachloride, dichloroethane, and combinations thereof.

In a further embodiment of any of the foregoing embodiments, the Lewis acid is selected from the group consisting of perchloric acid, sulfuric acid, alkylsulfonic acid, methane sulfonic acid, arylsulfonic acid, p-toluene sulfonic acid, and combinations thereof.

In a further embodiment of any of the foregoing embodiments, the solvent is a nitrated hydrocarbon selected from the group consisting of nitromethane, nitroethane, and combinations thereof.

In a further embodiment of any of the foregoing embodiments, the Lewis acid is selected from the group consisting of perchloric acid, sulfuric acid, alkylsulfonic acid, methane sulfonic acid, arylsulfonic acid, p-toluene sulfonic acid, and combinations thereof.

In a further embodiment of any of the foregoing embodiments, the solvent is selected from the group consisting of hexane, heptane, cyclohexane, isooctane, tolulene, benzene, dichloromethane, chloroform, carbon tetrachloride, dichloroethane, nitromethane, nitroethane, and combinations thereof.

In a further embodiment of any of the foregoing embodiments, the Lewis acid is selected from the group consisting of perchloric acid, sulfuric acid, alkylsulfonic acid, methane sulfonic acid, arylsulfonic acid, p-toluene sulfonic acid, and combinations thereof.

In a further embodiment of any of the foregoing embodiments, the Lewis acid includes methane sulfonic acid.

In a further embodiment of any of the foregoing embodiments, the removing includes removing the Lewis acid by filtration.

In a further embodiment of any of the foregoing embodiments, the removing includes removing the Lewis acid by quenching with a basic pH solution.

In a further embodiment of any of the foregoing embodiments, the removing includes removing the solvent by evaporation.

In a further embodiment of any of the foregoing embodiments, a ratio of the solvent in milliliters to the Lewis acid in milligrams is from 0.05 to 0.5.

In a further embodiment of any of the foregoing embodiments, the Delta-8-tetrahydrocannabinol is of at least 80% purity by weight with regard to total weight of CBD and tetrahydrocannabinol.

In a further embodiment of any of the foregoing embodiments, the mixture is agitated for 0.1 hours to 24 hours.

### DETAILED DESCRIPTION

Disclosed herein is a cannabis conversion method for converting cannabidiol (CBD) to Delta-8-tetrahydrocannabinol (Delta-8-THC). As will be appreciated from this disclosure, the disclosed method can be used to facilitate relatively short reaction times, high yields, and high purity Delta-8-THC.

An exemplary method according to this disclosure initially includes providing a starting material that includes CBD. For instance, the CBD is provided as a pre-prepared high purity CBD oil or as a component of a cannabis oleoresin, which may typically contain, by weight, from 1% to 90% CBD. The starting material is typically in a crude form and thus does not contain any solvents. Alternatively, if not prepared, the starting material may be prepared in a known manner by such known techniques as extraction from cannabis plant material, decarboxylation, etc.

The starting material is then combined with a solvent and an acid to form a mixture. The mixture need not be heated above ambient temperature but is agitated, such as by stirring, for 0.1 hours to 24 hours. The amount of the solvent and the Lewis acid can be varied but are expected to be effective over a ratio of the solvent (in milliliters) to the Lewis acid (in milligrams) that is from 0.05 milliliters/milligrams to 0.5 milliliters/milligrams.

The Lewis acids catalyze fast conversion of CBD to form a product Delta-9-THC which is converted further to the more thermodynamically stable Delta-8-THC under the action of the same catalyst in the mixture, yielding a highly pure product—Delta-8-THC. For example, the yield of product Delta-8-THC is 80% or more and the product Delta-8-THC is of at least 80% purity by weight with regard to total weight of CBD and other isomer tetrahydrocannabinols.

The reaction is a highly selective conversion of CBD to delta-8 THC that is achieved by a high Lewis acidity of the reaction mixture and a solvent with a low Gutmann Donor Number (“GDM,” as understood from Cataldo F., A revision of the Gutmann donor numbers of a series of phosphoramides including TEPA. Eur. Chem. Bull., 2015, 4(2), 92-97. DOI: 10.17628/ECB.2015.4.92), such as dichloromethane, chloroform, hexane, toluene, etc., and a strong acid such as methane sulfonic acid. The combination of a low GDM solvent and the use of a strong acid allows cyclization of CBD in delta-9 THC for several minutes under the ascribed conditions. Additionally, due to the similar reaction requirements for isomerization of delta-9 THC to delta-8 THC, the same combination of solvent and acid rapidly and selectively isomerizes delta-9 THC to delta-8 THC during the following 20-60 minutes in the ascribed one pot process. The process is very clean, with high yields and is preparative i.e. allows the target delta-8 THC in kilogram scale.

In examples, a solvent with a low GDM is a hydrocarbon, such as hexane, heptane, cyclohexane, isooctane, tolulene, benzene, or combinations thereof. Additionally, or alternatively, a solvent with a low GDM is a halogenated hydrocarbon, such as dichloromethane, chloroform, carbon tetrachloride, dichloroethane, or combinations thereof. Additionally or alternatively, the solvent with low GDM is a nitrated hydrocarbon, such as nitromethane, nitroethane, or combinations thereof.

In further examples of any of the above examples, the Lewis acid is selected from perchloric acid, sulfuric acid, alkylsulfonic acid, methane sulfonic acid, arylsulfonic acid, p-toluene sulfonic acid, or combinations thereof.

The removal of the acids from the reaction mixture may include removing by filtration. For example, such filtration is conducted over silica gel, alumina, powders of sodium carbonate, sodium bicarbonate, or combinations thereof. Additionally, or alternatively, the Lewis acid is removed by quenching with a basic pH solution. For example, the basic pH solution is phosphate buffer, sodium bicarbonate solution or combinations thereof. Another example alternative is removal of the acid over anion exchange resin beads. The solvent is removed by evaporation.

The following examples demonstrate additional, non-limiting aspects of the disclosure.

### EXAMPLE 1

Starting material: Crystalline CBD from hemp extract (>96%, HPLC), dichloromethane (DCM)(ACS reagent grade), methane sulfonic acid (MSA)(reagent grade).

Protocol: 1.0 g (3.18 mmol) of CBD was placed in a 100 mL glass flask and 50 mL of DCM and 152 mg (1.59 mmol, 0.5 eq) of MSA were added. The reaction mixture was stirred at room temperature for 1 h, transferred to a separatory funnel, washed in turn with 3×20 mL of aqueous 7.5% NaHCO₃, saturated NaCl (25 mL). The quenched reaction mixture was dried over 2 g of anhydrous Na₂SO₄, evaporated in vacuum to yield 0.98 g (98%) of crude Delta-8-THC as pale pink foam. The reaction mixture was analyzed by TLC, UV-Vis, and HPLC.

### EXAMPLE 2

Starting material: Crude CBD from hemp extract (92%, HPLC), chloroform (ACS reagent grade), methane sulfonic acid (MSA) (reagent grade).

Protocol: 12.0 g (35.16 mmol) of CBD was placed in a 500 mL glass flask and 200 mL of chloroform (without stabilizers e.g. ethanol, methanol etc.) and 1.69 g (17.58 mmol, 0.5 eq) of MSA were added. The reaction mixture was stirred at room temperature for 1 h, transferred to a separatory funnel, washed in turn with 3×50 mL of aqueous 7.5% NaHCO₃, saturated NaCl (50 mL). The quenched reaction mixture was dried over 6 g of anhydrous Na₂SO₄ and evaporated in vacuum to yield 11.5 g (96%) of crude delta-8-THC as light brown oil. The reaction mixture was analyzed by TLC, UV-Vis, HPLC.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A cannabis conversion method comprising:
providing a starting material that includes cannabidiol (CBD);
combining the starting material with a solvent and a Lewis acid to form a mixture, the solvent and the Lewis acid reacting with the CBD to form a product Delta-8-tetrahydrocannabinol in the mixture; and
removing the solvent and the Lewis acid from the mixture, the removing leaving the product Delta-8-tetrahydrocannabinol.

2. The method as recited in claim 1, wherein the solvent is selected from the group consisting of hexane, heptane, cyclohexane, isooctane, tolulene, benzene, and combinations thereof.

3. The method as recited in claim 2, wherein the Lewis acid is selected from the group consisting of perchloric acid, sulfuric acid, alkylsulfonic acid, methane sulfonic acid, arylsulfonic acid, p-toluene sulfonic acid, and combinations thereof.

4. The method as recited in claim 1, wherein the solvent is a halogenated hydrocarbon selected from the group consisting of dichloromethane, chloroform, carbon tetrachloride, dichloroethane, and combinations thereof.

5. The method as recited in claim 4, wherein the Lewis acid is selected from the group consisting of perchloric acid, sulfuric acid, alkylsulfonic acid, methane sulfonic acid, arylsulfonic acid, p-toluene sulfonic acid, and combinations thereof.

6. The method as recited in claim 1, wherein the solvent is a nitrated hydrocarbon selected from the group consisting of nitromethane, nitroethane, and combinations thereof.

7. The method as recited in claim 6, wherein the Lewis acid is selected from the group consisting of perchloric acid, sulfuric acid, alkylsulfonic acid, methane sulfonic acid, arylsulfonic acid, p-toluene sulfonic acid, and combinations thereof.

8. The method as recited in claim 1, wherein the solvent is selected from the group consisting of hexane, heptane, cyclohexane, isooctane, tolulene, benzene, dichloromethane, chloroform, carbon tetrachloride, dichloroethane, nitromethane, nitroethane, and combinations thereof.

9. The method as recited in claim 1, wherein the Lewis acid is selected from the group consisting of perchloric acid, sulfuric acid, alkylsulfonic acid, methane sulfonic acid, arylsulfonic acid, p-toluene sulfonic acid, and combinations thereof.

10. The method as recited in claim 1, wherein the Lewis acid includes methane sulfonic acid.

11. The method as recited in claim 1, wherein the removing includes removing the Lewis acid by filtration.

12. The method as recited in claim 1, wherein the removing includes removing the Lewis acid by quenching with a basic pH solution.

13. The method as recited in claim 1, wherein the removing includes removing the solvent by evaporation.

14. The method as recited in claim 1, wherein a ratio of the solvent in milliliters to the Lewis acid in milligrams is from 0.05 to 0.5.

15. The method as recited in claim 1, wherein the Delta-8-tetrahydrocannabinol is of at least 80% purity by weight with regard to total weight of CBD and tetrahydrocannabinol.

16. The method as recited in claim 1, wherein the mixture is agitated for 0.1 hours to 24 hours.
